(19) United States
(12) Patent Application Publication
(10) Pub. No.: US 2007/0043088 A1
(43) Pub. Date: Feb. 22, 2007

(54) PROCESS FOR PREPARING LACIDIPINE

(76) Inventors: Eswaraiah Sajja, Hyderabad 500 072, A.P. (IN); Venkata Naga Kali Vara Prasada Raju Vetukuri, Hyderabad 500 072, A.P. (IN); Srinivas Reddy Ningam, Medak 502 318, A.P. (IN); Ravindra Vedantham, Hyderabad 500 025, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/464,541

(22) Filed: Aug. 15, 2006

Related U.S. Application Data
(60) Provisional application No. 60/766,589, filed on Jan. 30, 2006.

(30) Foreign Application Priority Data
Aug. 16, 2005 (IN) 1132/CHE/2005

(51) Int. Cl. C07D 211/82

(52) U.S. Cl. 514/355; 546/315

(57) ABSTRACT

A process for preparing lacidipine, comprising reacting a t-butoxy carbonyl methyl aryl phosphonium halide with o-phthalaldehyde, and further reacting a product comprising (E)-3-(2-formylphenyl)-2-propenoic acid, 1,1-dimethyl ethyl ester, without isolation, with ethyl-3-amino crotonate.

### INTRODUCTION TO THE INVENTION

The present invention relates to substantially pure lacidipine and a process for its preparation.

Chemically, lacidipine is (E)-4-[2-[3-(1,1-Dimethylethoxy)-3-oxo-1-propenyl]phenyl]-1,4-dihydro-2,6-dimethyl-3,5-pyridinedicarboxylic acid diethyl ester and has the structural formula as shown in Formula I.

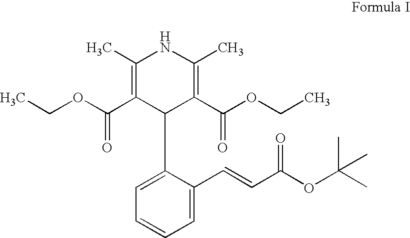

Lacidipine is a dihydropyridine calcium antagonist developed as a once-daily treatment for hypertension. Lacidipine works by relaxing and opening up the blood vessels. This allows blood to circulate more freely around the body, lowering blood pressure and allowing heart to work more efficiently.

Lacidipine is commercially available in the market in products sold under the trademark LACIMEN as tablet compositions.

Lacidipine, its related compounds and processes for their preparation have been described in U.S. Pat. Nos. 4,963,571 and 4,806,533, European Patent No. 245919, and GB 2164336.

U.S. Pat. No. 4,963,571 discloses a process for the preparation of lacidipine comprising reacting a bisaldehyde with a triphenyl phosphorane to give the corresponding olefin aldehyde. The olefin aldehyde in turn is reacted with an aminoester in the presence of a suitable catalyst to give lacidipine. The process suffers from low yields of the range of 24%, and involves chromatographic purifications of the intermediates making the process difficult to be scaled up.

The synthesis of lacidipine involves reactions in which undesired products such as regio isomers to the intermediates and to the final product are obtained. Therefore, the final product can be contaminated not only with the undesired products derived from the last synthetic step of the process but also with compounds that were formed in previous steps. These products should be removed from the final product in order to meet the ICH specifications for purity.

Regulatory authorities worldwide require that drug manufacturers isolate, identify and characterize the impurities in their products. Moreover, it is required to control the levels of these impurities in the final drug compound obtained by the manufacturing process and to ensure that the impurity is present in the lowest possible levels.

Hence, there is a constant need for processes for preparation of lacidipine that use a simple and commercially viable process whilst achieving the desired purity.

The present invention provides a process for the preparation of substantially pure lacidipine free of any process related impurities and also free of residual organic solvents. The process of the present invention can be practiced on an industrial scale, and also can be carried out without sacrifice of overall yield.

### SUMMARY OF THE INVENTION

The present invention relates to substantially pure lacidipine and a process for its preparation.

In one aspect, the invention provides substantially pure lacidipine.

In another aspect, the invention provides a process for the preparation of substantially pure lacidipine in high yields.

In an aspect, a process for the preparation of substantially pure lacidipine comprises:

a) condensation of a tertiary-butyl halo acetate with an aryl phosphine to give a t-butoxy carbonyl methyl aryl phosphonium halide;

b) condensation of a t-butoxy carbonyl methyl aryl phosphonium halide with o-phthalaldehyde to give (E)-3-(2-formylphenyl)-2-propenoic acid, 1,1-dimethyl ethyl ester; and

c) condensation of (E)-3-(2-formylphenyl)-2-propenoic acid, 1,1-dimethyl ethyl ester with ethyl 3-amino crotonate to give lacidipine.

Suitably, one or more sequential steps are carried out without isolating intermediate compounds. In one embodiment of the invention, step b) is carried out without isolating the intermediate, followed by isolation of the lacidipine.

In yet another aspect, the present invention provides a pharmaceutical composition comprising substantially pure lacidipine along with one or more pharmaceutically acceptable carriers, excipients or diluents.

An embodiment of a process for preparing lacidipine comprises:

reacting a t-butoxy carbonyl methyl aryl phosphonium halide with o-phthalaldehyde, and further reacting a product comprising (E)-3-(2-formylphenyl)-2-propenoic acid, 1,1-dimethyl ethyl ester with ethyl-3-amino crotonate.

Another embodiment of the invention comprises:

reacting t-butoxy carbonyl methyl triphenyl phosphonium bromide with o-phthalaldehyde, and further reacting a product comprising (E)-3-(2-formylphenyl)-2-propenoic acid, 1,1-dimethyl ethyl ester, without isolation, with ethyl-3-amino crotonate.

In either of these embodiments, the (E)-3-(2-formylphenyl)-2-propenoic acid, 1,1-dimethyl ethyl ester intermediate can be extracted, such as using a hydrocarbon or ether, before further reaction.

### BRIEF DESCRIPTION OF THE DRAWING

FIG. 1 is a schematic diagram of an ultrasonic flow cell that is useful in the practice of the present invention.

### DETAILED DESCRIPTION OF THE INVENTION

The present invention relates to substantially pure lacidipine and a process for its preparation.

In one aspect, the present invention provides a pure lacidipine, substantially free of the process related impurities.

By “pure lacidipine” it is meant that lacidipine or any of its pharmaceutically acceptable salts prepared in accordance with the present invention contains less than about 0.5%, or less than about 0.1%, of the corresponding impurities like the meta, para, and regio-isomers of lacidipine, carboxylic acid derivative, and the dimer impurities, as characterized by a high performance liquid chromatography (“HPLC”) chromatogram obtained from a mixture comprising the desired compound and the said impurities. The percentages herein refer to the area-% of the HPLC peaks representing the said impurities, as compared to the peak for lacidipine.

As used herein “meta isomer of lacidipine” refers to 4-[3-(2-tert-Butoxycarbonyl-vinyl)-phenyl]-2,6-dimethyl-1,4-dihydro-pyridine-3,5-dicarboxylic acid diethyl ester represented by Formula Ia;

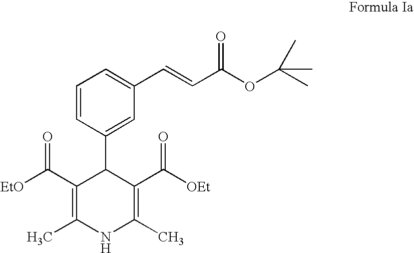

“Para isomer of lacidipine” refers to 4-[4-(2-tert-Butoxycarbonyl-vinyl)-phenyl]-2,6-dimethyl-1,4-dihydro-pyridine-3,5-dicarboxylic acid diethyl ester represented by Formula 1b;

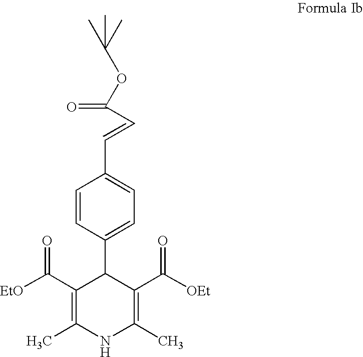

“Regio-isomer of lacidipine” refers to 4-[2-(2-tert-Butoxycarbonyl-vinyl)-phenyl]-3,6-dimethyl-1,4-dihydro-pyridine-2,5-dicarboxylic acid diethyl ester represented by Formula Ic;

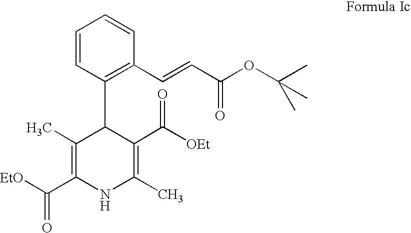

“Carboxylic acid derivative” refers to 4-(2-Carboxy-phenyl)-2,6-dimethyl-1,4-dihydro-pyridine-3,5-dicarboxylic acid diethyl ester represented by Formula IIb; and

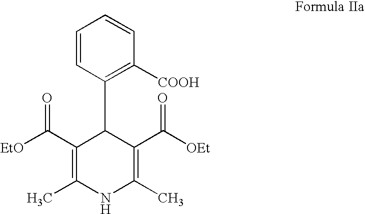

“Dimer impurity” refers to 3-[2-(2-tert-Butoxycarbonyl-vinyl)-phenyl]-acrylic acid tert-butyl ester represented by Formula IIb.

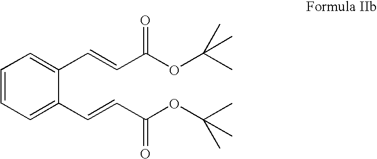

Lacidipine obtained using the process of the present invention also contains less than about 1000 ppm, or less than about 500 ppm, of individual residual organic solvents.

In another aspect, the invention provides a process for the preparation of pure lacidipine in high yields.

In an embodiment, the process for the preparation of pure lacidipine comprises:

a) condensation of a tertiary-butyl haloacetate with an aryl phosphine to give a t-butoxy carbonyl methyl aryl phosphonium halide; in an embodiment, tertiary-butyl bromoacetate of Formula II is reacted with triphenyl phosphine of Formula III to give t-butoxy carbonyl methyl triphenyl phosphonium bromide of Formula IV;

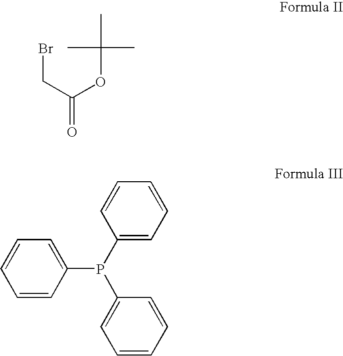

b) condensation of a t-butoxy carbonyl methyl aryl phosphonium halide with o-phthalaldehyde of Formula V to give (E)-3-(2-formylphenyl)-2-propenoic acid, 1,1-dimethyl ethyl ester of Formula VI; and

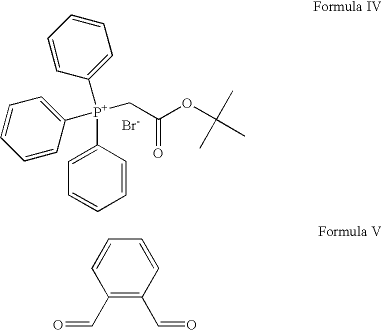

c) condensation of (E)-3-(2-formylphenyl)-2-propenoic acid, 1,1-dimethyl ethyl ester of Formula VI with ethyl 3-amino crotonate of Formula VII to give lacidipine of Formula I.

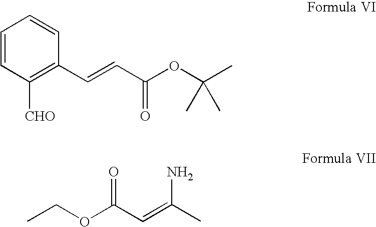

Suitably, one or more sequential steps are carried out without isolating intermediate compounds. In one embodiment of the invention, step b) is carried out without isolating the intermediate of Formula VI, followed by reaction with ethyl 3-amino crotonate and isolation of the compound of Formula I.

Step a) involves condensation of tertiary butyl halo acetate with aryl phosphine to give t-butoxy carbonyl methyl aryl phosphonium halide.

Tertiary butyl halo acetate that can be used includes tertiary butyl chlro acetate, tertiary butyl bromo acetate and tertiary butyl iodo acetate and the like.

Aryl phosphine that can be used include triphenyl phosphine, tri-ortho tolyl phosphines and the like.

Suitable solvents for conducting the reaction include, but are not limited to aprotic polar solvents such as N,N-dimethylformamide (DMF), dimethylsulfoxide (DMSO), N,N-dimethylacetamide, acetonitrile and the like; ethers such as tetrahydrofuran, 1,4-dioxane and the like; halogenated solvents such as dichloromethane, ethylene dichloride and the like; alcohols such as methanol, ethanol and the like; ketonic solvents such as acetone, methylisobutylketone and the like; hydrocarbons such as toluene and the like; or mixtures thereof

Suitable temperature for conducting the reaction may range from about 20° C. to about 70° C. The duration of maintenance of the reaction mixture at the reaction temperatures will be sufficient for reaction completion, and in some instances will be about 6 to 10 hours. The solid obtained after the completion of the reaction may optionally be filtered, or the reaction mass may be directly used in the next stage.

The product is suitably isolated from the reaction mass using techniques like filtration by gravity, or by suction, centrifugation, and the like. The crystals so isolated will carry a small proportion of occluded mother liquor containing a higher percentage of impurities. If desired the crystals can be washed on the filter with a solvent to wash out the mother liquor.

The solid obtained can optionally be dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods, times from about 1 to 20 hours frequently being suitable.

Step b) involves condensation of t-butoxy carbonyl methyl aryl phosphonium halide with o-phthalaldehyde of Formula V in the presence of a suitable base to give (E)-3-(2-formylphenyl)-2-propenoic acid, 1,1-dimethyl ethyl ester of Formula VI.

Suitable solvents which can be used for conducting the reaction include, but are not limited to: aprotic polar solvents such as DMF, DMSO, N,N-dimethylacetamide, acetonitrile and the like; ethers such as tetrahydrofuran, 1,4-dioxane and the like; halogenated solvents such as dichloromethane, ethylene dichloride and the like; alcohols such as methanol, ethanol and the like; ketonic solvents such as acetone, methylisobutylketone and the like; hydrocarbons such as toluene and the like; or mixtures thereof.

Suitable temperatures at which the reaction can be conducted can range from about −10° C. to 10° C.

Suitable bases which can be used include, but are not limited to organic bases such as methylamine, dimethylamine, triethylamine, di-isopropylethylamine, and the like, or inorganic bases like the carbonates and bicarbonates of alkali metals such as sodium carbonate, potassium carbonate, sodium bicarbonate and the like, hydroxides of alkali metals such as sodium hydroxide, potassium hydroxide and the like.

After completion of the reaction, aqueous layer is separated from the reaction mass and the organic layer is concentrated. The residue is dissolved in a suitable solvent and filtered to remove the undissolved by-product triphenyl phosphine oxide. The filtrate is taken into another reactor and can be directly used for proceeding to step c), or it can be distilled to isolate the product.

Selection of an appropriate solvent and temperature is critical for the effective removal of the by-product from the reaction mass. Retaining of excess or a part of the by-product in the reaction mass may lead to loss of yield in the next stage due to formation of impurities.

The solubility of the by-product triphenyl phosphine oxide when checked in solvents from various classes shows that the solubility is on the order of about 0.002 g/ml to about 0.0025 g/ml in hydrocarbons, 0.005 g/ml to about 0.01 g/ml in ethers, and about 0.04 g/ml to about 0.2 g/ml in alcohols and chlorohydrocarbons. Hence the classes of solvents which show the least solubility for the by-product are preferable for its separation from the reaction product.

Suitable solvents which can be used for the separation of by-product include, but are not limited to, hydrocarbons such as toluene, n-hexane, n-heptane, ethers such as diethyl ether, dimethyl ether, tetrahydrofuran, and the like, or mixtures thereof.

Suitable temperatures for the isolation of by-product range from about −10° C. to about 10° C. An optimized temperature can be selected by one skilled in the art to ensure that the byproduct is removed efficiently and at the same time minimize loss of the final product depending on the solvent used.

The percentage of the by-product triphenyl phosphine oxide present in the lacidipine product generally is less than about 0.5 area-%, or less than about 0.1 area-%, by HPLC.

The reaction mass obtained can be directly used for proceeding to step c), or it can be distilled to isolate the product.

Proceeding to the next stage with the organic layer without isolation of the product leads to improved yields and higher purity of the product at this stage. The intermediate isolated at this stage is somewhat unstable and hence leads to loss of yield due to formation of impurities when isolated and exposure to atmospheric conditions.

Step c) involves condensation of (E)-3-(2-formylphenyl)-2-propenoic acid, 1,1-dimethyl ethyl ester of Formula VI with ethyl 3-amino crotonate of Formula VII in the presence of a suitable acid to give lacidipine of Formula I.

Suitable solvents which can be used include, but are not limited to aprotic polar solvents such as DMF, DMSO, N,N-dimethylacetamide, acetonitrile and the like; ethers such as tetrahydrofuran, 1,4-dioxane and the like; halogenated solvents such as dichloromethane, ethylene dichloride and the like; alcohols such as methanol, ethanol, isopropanol and the like; ketonic solvents such as acetone, methylisobutylketone and the like; hydrocarbons such as toluene and the like; or mixtures thereof.

Suitable acids which can be used for the reaction include, but are not limited to organic acids such as acetic acid, trifluoroacetic acid, paratoluene sulfonic acid, oxalic acid, formic acid, and the like or mixtures thereof.

Suitable temperatures for conducting the reaction may range from about −10° C. to about 1° C.

The product isolated may optionally be purified by slurrying or recrystallization or a combination thereof in a suitable solvent. Recrystallization involves providing a solution of crude lacidipine in a suitable solvent and then crystallizing a more pure solid from the solution.

Suitable solvents in which lacidipine can be dissolved for purification include but are not limited to dimethylformamide, tetrahydrofuran, C₁ to C₅ straight chain carboxylic acids such as acetic acid, propanoic acid; alcohols such as ethanol, methanol, isopropanol, ethers such as ethers such as tetrahydrofuran, 1,4-dioxane, ethyl acetate and the like, water and mixtures thereof.

The concentration of the lacidipine in the solvent can range from 40 to 80% or more. The solution can be prepared at an elevated temperature if desired to achieve the desired concentration. Any temperature is acceptable for the dissolution as long as a clear solution of the lacidipine is obtained and is not detrimental to the drug substance chemically or physically. The solution may be brought down to room temperature for further processing if required or an elevated temperature may be used. A higher temperature will allow the precipitation of solutions with higher concentrations of lacidipine resulting in better economies of manufacture.

The recrystallization process may be conveniently carried out y using a flow cell, such as is described in our copending International Application No. PCT/US2006/013783.

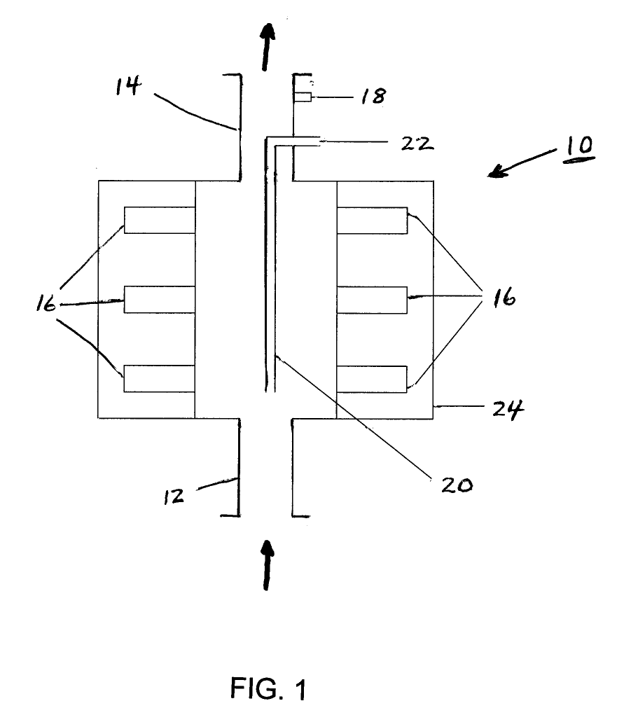

FIG. 1 is a cross-sectional diagram of an embodiment of an ultrasound flow cell 10. The flow cell comprises an inlet 12 and an outlet 14, for establishing a flow, in the direction of the arrows, through the cell of a first fluid that is introduced into inlet 12. The number, locations and dimensions of the inlets and outlets may vary according to the requirements of an application, and fluid flow for an embodiment can be opposite the direction shown, in which event the positions of inlet 12 and outlet 14 would be reversed. On the outer surface of flow cell 10 are mounted multiple ultrasonic transducers 16 that supply ultrasonic radiation into the contents of the flow cell, the transducers being electrically connected to a suitable external power source (not shown). The number and physical arrangement of the transducers around the flow cell can vary according to the process requirements. A temperature sensor 18 is optionally attached to outlet 14 to observe and control the temperature of the removed product from the outlet. Similar sensors may also optionally be attached to the inlet system and at various locations on the flow cell, as desired. A dip tube 20 having an inlet 22 is used to supply a second fluid containing a reactant into the flow cell. The number, diameters and materials of construction of the dip tube may vary with the process requirements. Dip tube 20 may further be perforated at a point where it is desired to introduce reactant into the flow cell interior and therefore does not necessarily have an open lower end for delivery of the reactant. The flow cell optionally is provided with an outer cover 24, for protection from the environment or for provision of heating, cooling, or insulation to the cell.

In operation, a first fluid comprising a reactive substance is introduced into inlet 12 and flows toward outlet 14. A second fluid comprising a different reactive substance is introduced into inlet 22 of dip tube 20 and exits the dip tube into the flow of the first reactive substance. Simultaneously, ultrasonic radiation having a desired frequency and power can be applied to flow cell 10 through transducers 16 to influence a reaction that takes place between the reactive substances.

Lacidipine obtained using the process of the present invention is substantially free of the process related impurities. It contains less than about 0.15 area-%, or less than about 0.1 area-%, or less than about 0.05 area-%, as determined by HPLC, of any of the following impurities:
i) 4-[3-(2-tert-Butoxycarbonyl-vinyl)-phenyl]-2,6-dimethyl-1,4-dihydro-pyridine-3,5-dicarboxylic acid diethyl ester represented by Formula Ia.
ii) 4-[4-(2-tert-Butoxycarbonyl-vinyl)-phenyl]-2,6-dimethyl-1,4-dihydro-pyridine-3,5-dicarboxylic acid diethyl ester represented by Formula Ib.
iii) 4-[2-(2-tert-Butoxycarbonyl-vinyl)-phenyl]-3,6-dimethyl-1,4-dihydro-pyridine-2,5-dicarboxylic acid diethyl ester represented by Formula Ic.
iv) 4-(2-Carboxy-phenyl)-2,6-dimethyl-1,4-dihydro-pyridine-3,5-dicarboxylic acid diethyl ester represented by Formula IIa.
v) 3-[2-(2-tert-Butoxycarbonyl-vinyl)-phenyl]-acrylic acid tert-butyl ester represented by Formula IIb.

Lacidipine obtained using the process of the present invention contains less than about 1000 ppm, or less than about 500 ppm of isopropanol, and less than about 1000 ppm, or less than about 500 ppm, of any other organic solvent.

In yet another aspect, the present invention provides a pharmaceutical composition comprising pure lacidipine along with one or more pharmaceutically acceptable carriers, excipients or diluents.

The pharmaceutical composition comprising pure lacidipine along with one or more pharmaceutically acceptable carriers of this invention may further formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the present invention include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

In the compositions of present invention lacidipine is a useful active ingredient in the range of 0.5 mg to 50 mg, or 1 mg to 25 mg.

The following examples further describe certain specific aspects and embodiments of the invention, and are not to be construed as limiting the scope of the invention.

### EXAMPLE 1

### PREPARATION OF TERTIARY BUTOXY CARBONYL METHYL TRIPHENYL PHOSPHONIUM BROMIDE

30 liters of toluene was taken into a reactor and 3.2 kg of triphenyl phosphine was added to it under a nitrogen atmosphere. Another 2 liters of toluene was added to the reactor and stirred for about 25 minutes. The reaction mass was then heated to about 60° C. and 2.9 kg of tertiary-butyl bromo acetate was added to the reaction mass at 60° C. Temperature of the reaction mass was then raised to 62° C. and maintained for 4 hours. Reaction completion was checked using thin layer chromatography. After the reaction was completed, the reaction mass was cooled to about 35° C. and filtered. The filtered cake was washed with 9.5 liters of toluene. The wet solid was dried at 60° C. for 4 hours to yield 5.36 kg (96% yield) of the title compound.

Purity by HPLC: 99.1 area-%.

### EXAMPLE 2

### PREPARATION OF DIETHYL (E)-4-[2-[(TERT-BUTOXY CARBONYL) VINYL]PHENYL]-1,4-DIHYDRO-2,6-DIMETHYL PYRIDINE-3,5-DICARBOXYLATE (FORMULA I)

18 liters of dichloromethane was taken into a reactor and 5 kg of tertiary-butoxy carbonyl methyl triphenyl phosphonium bromide (obtained using a similar process as described in Example 1) was added to it. 2.05 kg of ortho-phtalaldehyde was added to the reaction mass and another 1 liter of dichloromethane was added to it. The reaction mass was stirred for about 15 minutes and then cooled to about −5° C. A solution of 2.65 kg of sodium hydroxide flakes in 5 liters of water at about 25° C. was added to the reaction mass at −3° C. and maintained at −3° C. for 2.5 hours. Reaction completion was checked using thin layer chromatography. After the reaction was completed, the temperature of the reaction mass was raised to 25° C. and stirred for 30 minutes. The organic layer was separated from the reaction mass and distilled without vacuum at a temperature of 52° C. The residue obtained was maintained at 52° C. for 15 minutes. 35 liters of n-heptane was added to the reaction mass at 52° C. 4 liters of the n-heptane was distilled off from the reaction mass under a vacuum of 500 mm Hg at 63° C. The reaction mass was then cooled to 35° C. and maintained for 1.5 hours. The reaction mass was then filtered under vacuum to remove the undissolved material. The filtered cake was washed with 7.5 liters of n-heptane. The filtrate was taken into another reactor and the solvent was distilled off completely under a vacuum of 610 mm Hg and at 68° C. The reaction mass was then cooled to 33° C. and 11.5 liters of isopropanol was charged into it. The reaction mass was then cooled to −7° C. A solution of 4.25 kg of ethyl-3-aminocrotonate in 12.5 liters of isopropanol was added to the reaction mass at −7° C. 2.8 liters of trifluoroacetic acid was added to the reaction mass at −7° C. followed by addition of 1 liter of isopropanol. The reaction mass was maintained at −7° C. for 2 hours 45 minutes. Reaction completion was checked using thin layer chromatography. A solution of 2.6 kg of sodium bicarbonate in 50 liters of water was added to the reaction mass 0° C. and 25 liters of ethyl acetate was added to it. The temperature of the reaction mass was heated to 25° C. and stirred for 20 minutes. The aqueous layer was separated and extracted with 12.5 liters of ethyl acetate. The combined organic layer was taken into a separate reactor and the solvent was distilled off atmospherically at 81° C. The residue was distilled off completely, and then 5 liters of isopropanol was added to it. The reaction mass was distilled off completely and again 5 liters of isopropanol was added to the residue obtained. The reaction mass was again distilled off completely and finally the residue was dissolved in 63 liters of isopropanol by heating to 85° C. to get clear dissolution. The reaction mass was then cooled to 2° C. and maintained for 1.5 hours. The isolated material was filtered and washed with 2.5 liters of isopropanol. The wet material was dried at 50° C. for 30 minutes.

The dry material was taken into another reactor and 23 liters of isopropanol was added to it. The reaction mass was heated to 65° C. and maintained for 30 minutes. The reaction mass was then cooled to 2° C. and maintained for 1 hour. The reaction mass was filtered and washed with 1.5 liters of isopropanol. The wet solid was dried at a temperature of 60° C. and a vacuum of 630 mm Hg for 5 hours to yield 2.04 kg (yield: 40.9%) of the title compound.

Triphenyl phosphine oxide content: less than 0.0011 area-%.

### EXAMPLE 3

### PURIFICATION OF DIETHYL (E)-4-[2-[(TERT-BUTOXY CARBONYL) VINYL]PHENYL]-1,4-DIHYDRO-2,6-DIMETHYL PYRIDINE-3,5-DICARBOXYLATE (FORMULA I)

500 liters of demineralized water was taken into a reactor and cooled to 3° C. 47.3 liters of isopropanol was taken into a second reactor and 1.97 kg of lacidipine crude was dissolved in it. The solution was heated to 61° C. and maintained for 30 minutes. The demineralized water from the first reactor was fed with a flow rate of 60±2 liters per hour through a flow cell, as described above and in FIG. 1 and having an interior volume of 2 liters, for 30 minutes. The ultrasound system of the flow cell was then switched on, the feeding of water was continued at a temperature of 3° C., and simultaneously the solution of lacidipine prepared in the second reactor was fed from the reactor at 61° C. through the flow cell dip tube having outlet perforations, at a rate of 10.5 liters per hour. The resultant slurry obtained from the cell outlet was continuously centrifuged. After completion of the addition of the solution of lacidipine, the feeding of demineralized water was continued and simultaneously 10 liters of isopropanol was added through the dip tube to flush out the retained material. Feeding of the demineralized water was continued to purge the material from the flow cell and the resultant slurry was centrifuged for 1 hour for removal of liquid from the solid. The solid was then dried under a vacuum of 680 mm Hg and a temperature of 83° C. for 11.5 hours. The dried material was sifted through a 60 mesh sieve to get 1.78 kg (yield 90%) of the title compound.

Purity by HPLC: 99.9%

Meta isomer content: Less than 0.1 area-%.

Para isomer content: Less than 0.1 area-%.

Regio isomer content: Less than 0.1 area-%.

Carboxylic acid derivative content: Less than 0.1 area-%.

Dimer impurity content: Less than 0.1 area-%.

### EXAMPLE 4

### PREPARATION OF DIETHYL (E)-3-(2-FORMYLPHENYL)-2-PROPENOIC ACID, 1,1-DIMETHYL ETHYL ESTER OF FORMULA VI BY ISOLATION OF THE BY-PRODUCT TRIPHENYL PHOSPHINE OXIDE IN DIFFERENT SOLVENTS

Diethyl (E)-3-(2-formylphenyl)-2-propenoic acid, 1,1-dimethyl ethyl ester of Formula VI was prepared using a process similar to that described in Example 2, and different solvents were used for extraction of the intermediate. Different solvents were used to check the amount of the by-product not being extracted (not dissolved) from the reaction mass. The results are tabulated in Table 1. The results show that the use of n-heptane results in reduced extraction of the by-product.

TABLE 1

| Solvent Used | % of |
|---|---|
| for Intermediate | By-product |
| Extraction | Not Extracted |
| Diethyl ether | 80.5 |
| Diethyl ether | 83.3 |
| n-Hexane | 83.3 |
| n-Hexane | 82.0 |
| Cyclohexane | 93.1 |
| Cyclohexane | 92.0 |
| n-Heptane | 96.6 |
| n-Heptane | 98.3 |

### EXAMPLE 5

### DETERMINATION OF IMPURITIES IN LACIDIPINE

HPLC Method 1: Concentration of triphenyl phosphine oxide and compounds of Formula Ic and IIb in lacidipine are determined using HPLC as described in Table 2.

TABLE 2
HPLC method for detecting the level of the impurities.

| | |
|---|---|
| Chromatographic | A liquid chromatograph equipped with |
| conditions: | variable wavelength UV detector |
| | and integrator. |
| Column and Packing: | Lichrosphere CN 250 × 4.0 mm, 5 μm |
| Flow rate: | 1.0 ml/minute |
| Column oven | 27° C. |
| temperature: | |
| Wavelength: | 220 nm |
| Injection volume: | 10 μl |
| Run time: | 60 minutes |
| Elution: | Gradient |
| Diluent: | Acetonitrile |

| | | |
|---|---|---|
| | Time in minutes | % B Concentration |
| Gradient profile: | 0.1 | 05 |
| | 5 | 05 |
| | 10 | 10 |
| | 35 | 60 |
| | 50 | 60 |
| | 55 | 05 |
| | 60 | 05 |

| IMPURITY NAME | RRT |
|---|---|
| Triphenyl phosphine oxide | 0.66 |
| Meta isomer of lacidipine | 1.01 |
| Carboxylic acid derivative | 0.37 |
| Lacidipine | 1.0 |

HPLC method 2: Concentrations of the compounds of Formula Ia, Ib, and IIb in lacidipine are determined using HPLC as described in Table 3.

TABLE 3
HPLC method for detecting the level of the impurities.

| | |
|---|---|
| Chromatographic | A liquid chromatograph equipped with variable |
| conditions: | wavelength UV detector and integrator. |
| Column and Packing: | Waters Spherisorb CN 250 × 4.6 mm, 5 μm |
| Flow rate: | 1.0 ml/minute |
| Column oven | 27° C. |
| temperature: | |
| Wavelength: | 240 nm. |
| Injection volume: | 20 μl |
| Run time: | 50 minutes |
| Elution: | Isocratic |
| Diluent: | Mobile phase:ethanol = 9:1 |
| Mobile phase: | 970 ml of n-hexane and 30 ml of absolute |
| | ethanol, mixed well and sonicated. |

| IMPURITY NAME | RRT |
|---|---|
| Dimer impurity | 0.15 |
| Para isomer of lacidipine | 1.28 |
| Regio isomer of lacidipine | 0.41 |
| Lacidipine | 1.0 |

### EXAMPLE 6

### DETERMINATION OF RESIDUAL SOLVENTS IN LACIDIPINE

TABLE 4
Gas Chromatography method for detecting residual solvent content.

| | |
|---|---|
| Chromatographic conditions | The gas chromatography is equipped |
| | with a flame ionization detector. |
| Column: | AT-624 |
| Length | 30.0 meters |
| Internal diameter | 0.53 mm |
| Film Thickness | 3.0 μm |
| Injector temperature | 140° C. |
| Detector temperature | 260° C. (FID) |
| Mode of injection | Split |
| Split ratio | 1:5 |
| Carrier gas | Helium |
| Flow rate | 2.2 psi |
| Injection volume | 1.0 μl |
| Diluent | Dimethyl sulfoxide |

Oven temperature program: The column temperature is held at 40° C. for 10 minutes, then increased to 140° C. at a rate of 8° C. per minute, then held at 140° C. for 10 minutes. Again the temperature is increased to 250° C. at a rate of 35° C. and held for 10 minutes at 250° C.

### EXAMPLE 7

### PREPARATION OF 4-[3-(2-TERT-BUTOXYCARBONYL-VINYL)-PHENYL]-2,6-DIMETHYL-14-DIHYDRO-PYRIDINE-3,5-DICARBOXYLIC ACID DIETHYL ESTER (FORMULA Ia)

100 ml of dichloromethane, 25 g of t-butoxy carbonyl methyl triphenyl phosphonium bromide and 10.2 g of isophthalaldehyde were taken into a clean and dry round bottom flask and the resultant reaction mass was stirred for 20 minutes for homogenous solution. The reaction mass was then cooled to 3° C. followed by addition of a mixture of 13.2 g of sodium hydroxide and 20 ml of water in 40 minutes. The organic layer was separated and dried on 5 g of anhydrous sodium sulphate. The organic layer was then distilled off completely at about 40° C. 50 ml of n-heptane was added to the resultant residue and was stirred at 28° C. for 1 hour followed by filtration of separated solid and the solid was washed with 25 ml of n-heptane. The filtrate was taken into a clean and dry round bottom flask and the solvent was distilled off completely at about 62° C. to afford 12 g of intermediate residue.

10 g from the above obtained residue and 50 ml of isopropyl alcohol were charged in clean and dry round bottom flask and cooled to −5° C. 9.8 g of trifluoroactetic acid was added to the reaction mass, followed by addition of a mixture of 16.6 g of ethyl-3-aminocrotonate and 50 ml of isopropyl alcohol in 35 minutes. The resultant reaction mass was stirred for 1 hour followed by addition of a solution of 10.4 g of sodium bicarbonate in 130 ml of water in 50 minutes. 100 ml of ethyl acetate was added to the reaction suspension and was stirred for 30 minutes. The organic layer was separated and the aqueous layer was extracted with 30 ml of ethyl acetate. The combined organic layer was distilled from the organic layer at 60° C. The resultant residue was cooled to 30° C. 40 ml of isopropyl alcohol was added to the above residue and the reaction mass was cooled to 0° C. The reaction mass was stirred at 0° C. for 25 minutes. Separated solid was filtered followed by drying the solid at about 65° C. for about 3 hours to afford 6 g of the title compound.

### EXAMPLE 8

### PREPARATION OF 4-[4-(2-TERT-BUTOXYCARBONYL-VINYL)-PHENYL]-2,6-DIMETHYL-1,4-DIHYDRO-PYRIDINE-3,5-DICARBOXYLIC ACID DIETHYL ESTER (FORMULA Ib)

100 ml of dichloromethane, 24 g of t-butoxy carbonyl methyl triphenyl phosphonium bromide and 10 g of terephalaldehyde were taken into a clean and dry round bottom flask and the resultant reaction mass was stirred for 15 minutes for attaining a homogenous solution. The reaction mass was then cooled to 0° C. followed by addition of a solution of 13.6 g of sodium hydroxide in 20.6 ml of water. The organic layer was separated and dried over 5 g of anhydrous sodium sulphate. The organic layer was then distilled completely at 40° C. The resultant residue was cooled to about 30° C. and then 50 ml of cyclohexane was added to the resultant residue followed by further cooling to about 0° C. The reaction mass was stirred at 0° C. for 1 hour followed by filtration of separated solid. The filtered solid was washed with 25 ml of cyclohexane. The filtrate was taken into a clean and dry round bottom and distilled off completely at about 65° C. to afford 11.6 grams of residue.

10 g of the above residue and 50 ml of isopropyl alcohol were taken into a clean and dry round bottom flask followed by cooling to about −5° C. 9.8 g of trifluoroactetic acid was added to it followed by addition of a solution of 16.6 g of ethyl-3-aminocrotonate in 50 ml of isopropyl alcohol. The resultant reaction suspension was stirred for 1 hour followed by addition of a solution of 10.4 g of sodium bicarbonate in 130 ml of water. 100 ml of ethyl acetate was added to the reaction suspension and was stirred for 15 minutes followed by separation of organic layer. The aqueous layer was extracted with 50 ml of ethyl acetate. The combined organic layer was washed with 100 ml of water. The organic layer was distilled at 65° C. The residue obtained was cooled to 28° C. 100 ml of petroleum ether was added to the above crude solid and was stirred for 15 minutes. The separated solid was filtered and washed with 20 ml of petroleum ether. The wet solid was dried at 65 for 3 hours to afford 17 g of the title compound.

### EXAMPLE 9

### PREPARATION OF 4-(2-CARBOXY-PHENYL)-2,6-DIMETHYL-1,4-DIHYDRO-PYRIDINE-3,5-DICARBOXYLIC ACID DIETHYL ESTER (FORMULA IIa)

50 ml of isopropyl alcohol and 10 g of 2-formylbenzoic acid were taken into a clean and dry round bottom flask followed by cooling to about −10° C. A solution of 27 g of ethyl-3-aminocrotonate in 50 ml of isopropyl alcohol was added slowly to the reaction mass followed by addition of 16 g of trifluoroacetic acid. The resultant reaction mass was stirred for 45 minutes followed by quenching the reaction mass by the addition of a solution of 21.5 g of sodium bicarbonate in 300 ml of water. The temperature of the reaction mass was raised to 29° C. followed by addition of 100 ml of ethyl acetate. The reaction mass was stirred for 20 minutes. The organic layer was separated and the aqueous layer was extracted with 30 ml of ethyl acetate. The combined organic layer was distilled at 60° C. under a vacuum of 300 mm/Hg. 30 ml of isopropyl alcohol was added to it and was cooled to 3° C. The reaction mass was stirred at 3° C. for 45 minutes. 300 ml of precooled water (to about 5-10° C.) was added and was stirred for about 1 hour. The separated solid was filtered and the solid was washed with 10 ml of water followed by drying the solid at about 65° C. under a vacuum of 300 mm Hg for about 2 hours to afford 8 g of the title compound.

### EXAMPLE 10

### PREPARATION OF 4-[2-(2-TERT-BUTOXYCARBONYL-VINYL)-PHENYL]-3,6-DIMETHYL-1,4-DIHYDRO-PYRIDINE-2,5-DICARBOXYLIC ACID DIETHYL ESTER (FORMULA Ic)

15 ml of acetic acid and 5 g of (E)-3-(2-formylphenyl)-2-propenoic acid 1,1-dimethylethyl ester were taken into a clean and dry round bottom and stirred for 10 minutes. 5.6 g of ethyl-3-aminocrotonate was added to it and stirred for 6 hours followed by quenching of the reaction mass by the addition of reaction mass to 25 ml of water. The reaction mass was extracted with 50 ml of ethyl acetate. The ethyl acetate layer was washed with 10 ml of 5% aqueous sodium bicarbonate solution. The organic layer was dried on 2.5 g of sodium sulphate followed by distillation of solvent completely at about 60° C. under a vacuum of 300 mm Hg. 15 ml of petroleum ether was added to the residue and was cooled to about 5° C. The reaction mass was maintained at 5° C. for 1 hour. The solvent was decanted and again 20 ml of methyl tertiarybutyl ether was added to it and stirred for 10 minutes. 100 ml of petroleum ether was added to it and the mixture was allowed to stand overnight. Resultant suspension was stirred for 40 minutes followed by filtration of separated solid. The solid was washed with 10 ml of petroleum ether to afford 3.4 grams of the title compound.

### EXAMPLE 11

### PREPARATION OF 3-[2-(2-TERT-BUTOXYCARBONYL-VINYL)-PHENYL]-ACRYLIC ACID TERT-BUTYL ESTER (FORMULA IIb)

250 ml of 1,4-dioxane, 25 g of ortho-pthalaldehyde and 85.3 g of t-butoxy carbonyl methyl triphenyl phosphonium bromide were taken into a clean and dry round bottom flask and stirred for 30 minutes. Resultant reaction mixture was cooled to 20° C. followed by stirring for 15 minutes. 50 ml of 48% aqueous sodium hydroxide solution was added to the reaction mass and stirred for 45 minutes. Reaction suspension was heated to 50° C. and stirred for 1 hour followed by cooling to 30° C. The organic layer was separated and distilled completely at about 50° C. under a vacuum of 300 mm Hg. The residue obtained was cooled to 30° C. 75 ml of cyclohexane was added to it and the reaction mass was further cooled to 0° C. The reaction mass was maintained at 0° C. for 30 minutes. Separated solid was filtered and the solid was washed with 25 ml of cyclohexane. The filtrate was taken into another round bottom flask and distilled at 50° C. under a vacuum of 300 mm Hg followed by cooling the residue to about 30° C. 50 ml of petroleum ether was added to the above resultant residue and was stirred for 45 minutes. Separated solid was filtered and washed with 25 ml of petroleum ether followed by drying the obtained solid at 50° C. for 2 hours to afford 9 g of the title compound.

We claim:

1. A process for preparing lacidipine, comprising reacting a t-butoxy carbonyl methyl aryl phosphonium halide with o-phthalaldehyde, and further reacting a product comprising (E)-3-(2-formylphenyl)-2-propenoic acid, 1,1-dimethyl ethyl ester with ethyl-3-amino crotonate.

2. The process of claim 1, wherein a t-butoxy carbonyl methyl aryl phosphonium halide comprises t-butoxy carbonyl methyl triphenyl phosphonium bromide, t-butoxy carbonyl methyl triphenyl phosphonium chloride, or t-butoxy carbonyl methyl triphenyl phosphonium Iodide.

3. The process of claim 1, wherein a t-butoxy carbonyl methyl aryl phosphonium halide is prepared by reacting a tertiary butyl halo acetate with an aryl phosphine.

4. The process of claim 1, wherein (E)-3-(2-formylphenyl)-2-propenoic acid, 1,1-dimethyl ethyl ester is extracted with a hydrocarbon or ether, before further reacting.

5. The process of claim 4, wherein a hydrocarbon comprises n-heptane.

6. The process of claim 1, wherein lacidipine is purified by combining a solution of lacidipine in isopropanol with water, while applying ultrasound energy.

7. Lacidipine prepared by the process of claim 1, containing less than about 0.1 area-% by high performance liquid chromatography analysis of any of:
4-[3-(2-tert-Butoxycarbonyl-vinyl)-phenyl]-2,6-dimethyl-1,4-dihydro-pyridine-3,5-dicarboxylic acid diethyl ester;
4-[4-(2-tert-Butoxycarbonyl-vinyl)-phenyl]-2,6-dimethyl-1,4-dihydro-pyridine-3,5-dicarboxylic acid diethyl ester;
4-(2-Carboxy-phenyl)-2,6-dimethyl-1,4-dihydro-pyridine-3,5-dicarboxylic acid diethyl ester;
4-[2-(2-tert-Butoxycarbonyl-vinyl)-phenyl]-3,6-dimethyl-1,4-dihydro-pyridine-2,5-dicarboxylic acid diethyl ester;
3-[2-(2-tert-Butoxycarbonyl-vinyl)-phenyl]-acrylic acid tert-butyl ester; and
triphenyl phosphine oxide.

8. Lacidipine prepared by the process of claim 1, containing less than about 1000 ppm of a residual solvent, as determined by gas chromatography.

9. A pharmaceutical composition comprising lacidipine prepared by the process of claim 1 and one or more pharmaceutically acceptable carriers, excipients or diluents.

10. A process for preparing lacidipine, comprising reacting t-butoxy carbonyl methyl triphenyl phosphonium bromide with o-phthalaldehyde, and further reacting a product comprising (E)-3-(2-formylphenyl)-2-propenoic acid, 1,1-dimethyl ethyl ester, without isolation, with ethyl-3-amino crotonate.

11. The process of claim 10, wherein (E)-3-(2-formylphenyl)-2-propenoic acid, 1,1-dimethyl ethyl ester is extracted with a hydrocarbon, before further reacting.

12. The process of claim 10, wherein (E)-3-(2-formylphenyl)-2-propenoic acid, 1,1-dimethyl ethyl ester is extracted with an ether, before further reacting.

13. The process of claim 10, wherein (E)-3-(2-formylphenyl)-2-propenoic acid, 1,1-dimethyl ethyl ester is extracted with n-heptane, before further reacting.

14. The process of claim 10, wherein lacidipine is purified by combining a solution of lacidipine in isopropanol with water, while applying ultrasound energy.

15. Lacidipine prepared by the process of claim 1, containing less than about 0.1 area-% by high performance liquid chromatography analysis of any of:
4-[3-(2-tert-Butoxycarbonyl-vinyl)-phenyl]-2,6-dimethyl-1,4-dihydro-pyridine-3,5-dicarboxylic acid diethyl ester;
4-[4-(2-tert-Butoxycarbonyl-vinyl)-phenyl]-2,6-dimethyl-1,4-dihydro-pyridine-3,5-dicarboxylic acid diethyl ester;
4-(2-Carboxy-phenyl)-2,6-dimethyl-1,4-dihydro-pyridine-3,5-dicarboxylic acid diethyl ester;
4-[2-(2-tert-Butoxycarbonyl-vinyl)-phenyl]-3,6-dimethyl-1,4-dihydro-pyridine-2,5-dicarboxylic acid diethyl ester;
3-[2-(2-tert-Butoxycarbonyl-vinyl)-phenyl]-acrylic acid tert-butyl ester; and
triphenyl phosphine oxide.

16. Lacidipine prepared by the process of claim 1, containing less than about 1000 ppm of a residual solvent, as determined by gas chromatography.

17. A process for preparing lacidipine, comprising:
reacting t-butoxy carbonyl methyl triphenyl phosphonium bromide with o-phthalaldehyde, to form an intermediate (E)-3-(2-formylphenyl)-2-propenoic acid, 1,1-dimethyl ethyl ester;
extracting (E)-3-(2-formylphenyl)-2-propenoic acid, 1,1-dimethyl ethyl ester with a hydrocarbon or ether;
reacting extracted (E)-3-(2-formylphenyl)-2-propenoic acid, 1,1-dimethyl ethyl ester with ethyl-3-amino crotonate to form lacidipine; and
purifying lacidipine by combining a solution of lacidipine in isopropanol with water, while applying ultrasound energy.

18. The process of claim 17, wherein (E)-3-(2-formylphenyl)-2-propenoic acid, 1,1-dimethyl ethyl ester is extracted with n-heptane, before further reacting.

19. Lacidipine prepared by the process of claim 17, containing less than about 0.1 area-% by high performance liquid chromatography analysis of any of:
4-[3-(2-tert-Butoxycarbonyl-vinyl)-phenyl]-2,6-dimethyl-1,4-dihydro-pyridine-3,5-dicarboxylic acid diethyl ester;
4-[4-(2-tert-Butoxycarbonyl-vinyl)-phenyl]-2,6-dimethyl-1,4-dihydro-pyridine-3,5-dicarboxylic acid diethyl ester;
4-(2-Carboxy-phenyl)-2,6-dimethyl-1,4-dihydro-pyridine-3,5-dicarboxylic acid diethyl ester;
4-[2-(2-tert-Butoxycarbonyl-vinyl)-phenyl]-3,6-dimethyl-1,4-dihydro-pyridine-2,5-dicarboxylic acid diethyl ester;
3-[2-(2-tert-Butoxycarbonyl-vinyl)-phenyl]-acrylic acid tert-butyl ester; and
triphenyl phosphine oxide.

20. Lacidipine prepared by the process of claim 17, containing less than about 1000 ppm of a residual solvent, as determined by gas chromatography.
